(19) United States
(12) Patent Application Publication
(10) Pub. No.: US 2016/0000968 A1
(43) Pub. Date: Jan. 7, 2016

(54) PLACENTAL TISSUE GRAFTS AND IMPROVED METHODS OF PREPARING AND USING THE SAME

(71) Applicant: MiMedx Group, Inc., Marietta, GA (US)

(72) Inventors: Thomas J. Koob, Marietta, GA (US); Rebeccah J.C. Brown, Marietta, GA (US); Deborah L. Dean, Marietta, GA (US); Parker Petit, Marietta, GA (US)

(21) Appl. No.: 14/325,132

(22) Filed: Jul. 7, 2014

(51) Int. Cl. A61L 27/36

(52) U.S. Cl. CPC A61L 27/3604; A61L 27/3695

(57) ABSTRACT

Described herein are tissue grafts derived from the placenta. The grafts are composed of at least one layer of amnion which has been decellularized and at least one layer of chorion. Also described herein are methods for making and using the tissue grafts.

### BACKGROUND

Human placental membrane (e.g. amniotic membrane or tissue) has been used for various types of reconstructive surgical procedures since the early 1900s. The membrane serves as a substrate material, more commonly referred to as a biological dressing or patch graft. Such a membrane has also been used widely for ophthalmic procedures. Typically, such membrane is either frozen or dried for preservation and storage until needed for surgery.

Such placental tissue is typically harvested after an elective Cesarean surgery. The placenta is composed of the umbilical cord and amniotic sac. The amniotic sac, commonly referred to as the amniotic membrane, has two primary layers of tissue, amnion and chorion. Amnion tissue is the innermost layer of the amniotic sac and in direct contact with the amniotic fluid. The amniotic sac contains the amniotic fluid and protects the fetal environment. Histological evaluation indicates that the membrane layers of the amnion consist of a single layer of epithelial cells, thin reticular fibers (basement membrane), a thick compact layer, and a fibroblast layer. The fibrous layer of amnion (i.e., the basement membrane) contains collagen types IV, V, and VII, and cell-adhesion bio-active factors including fibronectin and laminins.

Amnion tissue provides unique grafting characteristics when used for surgical procedures, including providing a matrix for cellular migration/proliferation, providing a natural biological barrier, are non-immunogenic, and contains numerous bio-active molecules, which can be used as a membrane to assist in tissue regeneration and improved healing outcomes in numerous applications. The membrane has the capability to self-adhere or, in the alternative, is susceptible of being fixed in place using different techniques including fibrin glue or suturing. Such grafts, when properly prepared, can be stored at room temperature for extended periods of time, without need for refrigeration or freezing, until needed for a surgical procedure.

Known clinical procedures or applications for such amnion grafts include ocular reconstruction, burns, anti-adhesion applications, barrier membranes, and general wound care. Described herein are membranes utilizing amnion tissue as building blocks to build membranes well suited for use in a variety of application such as, for example, perioplastic surgery and other surgical applications involving human mucosa tissue. The grafts and methods described herein utilize these biological molecules to enhance the performance of the amniotic membrane.

### SUMMARY OF INVENTION

Described herein are laminated tissue grafts derived from placental tissues. The grafts are composed of a) at least one layer of modified amnion tissue where the epithelial layer (i.e., epithelial cells) has been substantially removed in order to expose the basement membrane layer to host cells and b) at least one layer of chorion. By removing the epithelial layer, cells from the host can more readily interact with the cell-adhesion bio-active factors located on top of and within the basement membrane. The fibroblast cellular layer is also substantially decellularized such that the amnion layer retains substantially no epithelial or fibroblast cells. In some embodiments, decellularization of the fibroblast layer can be achieved either by removal or substantial removal of the fibroblast cellular layer or by decellularizing the fibroblast layer which is otherwise retained in the amniotic membrane. The grafts are further composed of at least one layer of chorion. The chorion is optionally decellularized. The combination of an amnion layer having substantially all of its epithelial and fibroblast cells removed and a chorion layer provides for a tissue graft with significant amounts of growth factors without any interference from cellular components.

In one aspect, the invention relates to a laminated tissue graft comprising a first membrane which is a modified decellularized amnion membrane and a second membrane which is a chorion membrane layered adjacent to the first membrane. In one embodiment, the modified decellularized amnion membrane comprises a basement membrane from which the epithelial cells have been removed, and optionally a compact layer and/or a decellularized fibroblast layer. In one embodiment, the fibroblast layer has been removed from the amnion to expose the other side of the basement membrane (i.e. the side of the basement membrane adjacent to the compact layer or the fibroblast layer) or the compact layer. Thus, in some embodiments, the amnion membrane comprises a first side which is an exposed basement membrane and a second side which is a decellularized fibroblast layer, a compact layer, or the other side of the basement membrane. In one embodiment, the chorion membrane is decellularized.

In one embodiment, one or more membranes are layered adjacent to the first or second membrane. In one embodiment, the one or more additional membranes are selected from the group consisting of amnion, decellularized amnion, chorion, decellularized chorion, allograft pericardium, allograft acellular dermis, amniotic membrane, Wharton's jelly, and combinations thereof. In one embodiment, the laminated tissue graft further comprises at least one of a naturally occurring growth factor, a platelet concentrate, a bone marrow aspirate, stem cells, or an antibiotic.

Also described herein are methods for making and using the tissue grafts. The laminin structure of amnion tissue is nearly identical to that of native human tissue such as, for example, oral mucosa tissue. This includes high level of laminin-5, a cell adhesion bio-active factor shown to bind gingival epithelial cells, found throughout upper portions of the basement membrane.

The advantages of the invention will be set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below.

FIG. 1 is an overview flow chart of the process for making the tissue grafts described herein.

FIG. 2 is a perspective view of an exemplary drying fixture for making the tissue grafts described herein.

FIG. 3 is a top view of a tissue graft with perforations.

### DETAILED DESCRIPTION

Before the present articles and methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings^{.}

It must be noted that, as used in the specification and the appended claims, the singular forms “a,” “an” and “the” include plural referents unless the context clearly dictates otherwise. Thus, for example, reference to “a bioactive agent” includes mixtures of two or more such agents, and the like.

“Optional” or “optionally” means that the subsequently described event or circumstance can or cannot occur, and that the description includes instances where the event or circumstance occurs and instances where it does not. For example, the phrase “optionally cleaning step” means that the cleaning step may or may not be performed.

The term “cell” refers to intact cells regardless of whether such cells are viable or not. Preferably, the cells are intact but non-viable.

The term “substantially all” means that at least 90% of the cells are removed and, preferably, at least 95% of the cells are removed and, more preferably, at least about 99% of the cells are removed.

Titles or subtitles may be used in the specification for the convenience of a reader, which are not intended to influence the scope of the present invention. Additionally, some terms used in this specification are more specifically defined below.

### Tissue Grafts and Methods for Making Thereof

Described herein are laminated tissue grafts and methods of making and using thereof. In general, the tissue grafts are multilayered systems composed of one or more membranes laminated to a base amnion, where the epithelial layer of the base amnion has been substantially removed and substantially all of the cells in the fibroblast layer are removed. Alternatively, the fibroblast layer can simply be removed. In one embodiment, a chorion membrane is layered adjacent to the amnion membrane. In one embodiment, the chorion membrane is decellularized. In another embodiment, the chorion membrane is not decellularized.

In one aspect, the process for preparing the laminated tissue graft involves:
(a) obtaining a placenta from a subject, wherein the placenta comprises an amniotic membrane layer and a chorion membrane layer;
(b) separating the chorion membrane layer from the amnion membrane layer, wherein the amnion comprises epithelial cells adjacent to a basement membrane and a fibroblast layer on the opposite side of the amnion membrane;
(c) removing substantially all of the epithelial cells from the amnion layer to expose the basement membrane;
(d) decellularizing and/or removing the fibroblast layer from the amnion membrane;
(e) optionally decellularizing the chorion membrane;
(f) mounting the decellularized amnion membrane and the chorion membrane onto a surface of a drying fixture;
(g) optionally mounting one or more additional membranes on the amnion or chorion membrane to produce a layered tissue graft; and
(h) dehydrating the layered tissue graft on the drying fixture.

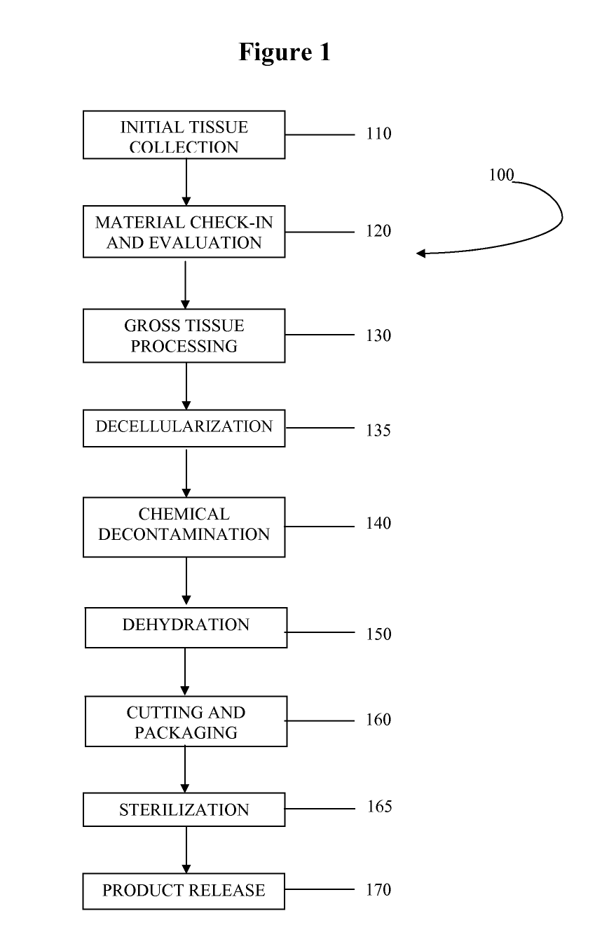

FIG. 1 depicts an overview (100) of the steps to harvest, process, and prepare placental material for later use as a tissue graft as disclosed. More detailed descriptions and discussion regarding each individual step will follow. Initially, the placenta tissue is collected from a consenting patient following an elective Cesarean surgery (step 110). The material is preserved and transported in a conventional tissue preservation manner to a suitable processing location or facility for check-in and evaluation (step 120). Gross processing, handling, and separation of the tissue layers then takes place (step 130). After the amnion is decellularized to expose the basement membrane and optionally the fibroblast layer is removed (step 135), acceptable tissue is decontaminated (step 140), layered and dehydrated (step 150), cut and packaged (step 160), tissue is sterilized using gamma radiation (step 165), and released (step 170) to the market for use by surgeons and other medical professionals in appropriate surgical procedures and for wound care. Each step is described in detail below.

### Initial Tissue Collection (Step 110)

The recovery of placenta tissue originates in a hospital, where it is collected during a Cesarean section birth. The donor, referring to the mother who is about to give birth, voluntarily submits to a comprehensive screening process designed to provide the safest tissue possible for transplantation. The screening process preferably tests for antibodies to the human immunodeficiency virus type 1 and type 2 (anti-HIV-1 and anti-HIV-2), hepatitis B surface antigens (HBsAg), antibodies to the hepatitis C virus (anti-HCV), antibodies to the human T-lymphotropic virus type I and type II (anti-HTLV-1 and anti-HTLV-II), CMV, and syphilis, using conventional serological tests. The above list of tests is exemplary only, as more, fewer, or different tests may be desired or necessary over time or based upon the intended use of the grafts, as will be appreciated by those skilled in the art.

Based upon a review of the donor's information and screening test results, the donor will be deemed either acceptable or not. In addition, at the time of delivery, cultures are taken to determine the presence of bacteria, for example, Clostridium or Streptococcus. If the donor's information, screening tests, and the delivery cultures are all satisfactory (i.e., do not indicate any risks or indicate acceptable level of risk), the donor is approved by a medical director and the tissue specimen is designated as initially eligible for further processing and evaluation.

Human placentas that meet the above selection criteria are preferably bagged in a saline solution in a sterile shipment bag and stored in a container of wet ice for shipment to a processing location or laboratory for further processing.

If the placenta tissue is collected prior to the completion or obtaining of results from the screening tests and delivery cultures, such tissue is labeled and kept in quarantine. The tissue is approved for further processing only after the required screening assessments and delivery cultures, which declare the tissue safe for handling and use, are satisfied and final approval is obtained from a medical director.

### Material Check-in and Evaluation (Step 120)

Upon arrival at the processing center or laboratory, the shipment is opened and verified that the sterile shipment bag/container is still sealed and in the coolant, that the appropriate donor paperwork is present, and that the donor number on the paperwork matches the number on the sterile shipment bag containing the tissue. The sterile shipment bag containing the tissue is then stored in a refrigerator until ready for further processing. All appropriate forms, including a tissue check-in form, chain of custody, and handling logs (not shown) are completed.

### Gross Tissue Processing (Step 130)

When the tissue is ready to be processed further, the sterile supplies necessary for processing the placenta tissue are assembled in a staging area in a controlled environment and are prepared for introduction into a controlled environment. If the controlled environment is a manufacturing hood, the sterile supplies are opened and placed into the hood using conventional sterile technique. If the controlled environment is a clean room, the sterile supplies are opened and placed on a cart covered by a sterile drape. All the work surfaces are covered by a piece of sterile drape using conventional sterile techniques, and the sterile supplies and processing equipment are placed on to the sterile drape, again using conventional sterile techniques.

Processing equipment is decontaminated according to conventional and industry-approved decontamination procedures and then introduced into the controlled environment. The equipment is strategically placed within the controlled environment to minimize the chance that the equipment will come in proximity to or inadvertently be contaminated by the tissue specimen.

Next, the placenta is removed from the sterile shipment bag and transferred aseptically to a sterile processing basin within the controlled environment. The sterile basin contains a hypertonic saline solution (e.g., 18% NaCl) that is at room or near room temperature. The placenta is gently massaged to help separate blood clots and to allow the placenta tissue to reach room temperature, which will make the separation of the amnion and chorion layers from each other, as discussed hereinafter, easier. After warming up to the ambient temperature (after about 10-30 minutes), the placenta is removed from the sterile processing basin and laid flat on a processing tray with the amniotic membrane layer facing down for inspection.

The placenta tissue is examined and the results of the examination are documented. The placenta tissue is examined for discoloration, debris or other contamination, odor, and signs of damage. The size of the tissue is also noted. A determination is made, at this point, as to whether the tissue is acceptable for further processing.

Next, if the placenta tissue is deemed acceptable for further processing, the amnion and chorion layers of the placenta tissue are carefully separated. In one aspect, the materials and equipment used in this procedure include a processing tray, 18% saline solution, sterile 4×4 sponges, and two sterile Nalgene jars. The placenta tissue is closely examined to find an area (typically a corner) in which the amnion layer can be separated from the chorion layer. The amnion appears as a thin, opaque layer on the chorion.

Prior to removal of the epithelium, the fibroblast layer is identified by gently contacting each side of the membrane with a piece of sterile gauze or a cotton tipped applicator. The fibroblast layer will stick to the test material. The amnion is placed into processing tray fibroblast layer down. Using a blunt instrument, a cell scraper or sterile gauze, any residual blood is also removed. This step must be done with adequate care, again, so as not to tear the amnion or chorion tissues. The cleaning of the amnion is complete once the amnion tissue is smooth and opaque-white in appearance. If the amnion tissue is cleaned too much, the jelly-like fibroblast layer can be removed. In some embodiments, any areas of the amnion cleaned too aggressively and appearing clear will be unacceptable and will ultimately be discarded.

Removal of Epithelial Layer from Amnion (Step 135)

The epithelial layer present on the amnion is substantially removed in order to expose the basement layer of the amnion. The significance of removing the epithelial layer is described below. The term “substantially removed” with respect to the amount of epithelium (which includes of course the epithelial cells) removed is defined herein as removing greater than 90%, greater than 95%, or greater than 99% of the epithelial cells from the amnion.

The epithelial layer can be removed by techniques known in the art. For example, the epithelial layer can be scraped off of the amnion using a cell scraper. In a preferred embodiment, the amnion is decellularized. That is, all or substantially all of the cells present in the amnion are removed or lysed. The term “substantially removed” with respect to the amount of cells removed is defined herein as removing greater than 90%, greater than 95%, or greater than 99% of the cells from the amnion. Techniques for decellularizing amnion include, but are not limited to, freezing the membrane, physical removal using a cell scraper, or exposing the membrane to nonionic detergents, anionic detergents, and nucleases.

In some embodiments, the fibroblast layer is removed or substantially removed from the amnion. The term “substantially removed” with respect to the amount of fibroblast layer removed is defined herein as removing greater than 90%, greater than 95%, or greater than 99% of the fibroblast layer from the amnion. The fibroblast layer may be removed before or after decellularization of the amnion membrane. Techniques for removing the fibroblast layer include, but are not limited to, physical removal using a cell scraper. Techniques for removing the cells in the fibroblast layer include exposing the membrane to nonionic detergents, anionic detergents, and/or nucleases. It is understood that exposing the epithelial or fibroblast side to such detergent solutions will in decellularization of the entire amnion layer as that layer is so thin and water absorbent, the solution applied will invariably penetrate through the entirety of the amnion thereby decellularizing the entirety of that layer.

The presence or absence of epithelial cells remaining on the amnion layer can be evaluated using techniques known in the art. The decellularized tissue can also be evaluated to determine that the basement membrane has not been compromised and remains intact. This step may be performed after completion of any processing step, or after the tissue has been dehydrated as described in the next section. For example, a representative sample graft is removed for microscopic analysis. The tissue sample is place onto a standard slide and 100 μl of Eosin Y stain is applied to the sample and allowed to set. The tissue sample is then examined under magnification. Cellular material will stain dark indicating the presence of cells. If no stained cells are present, decellularization has been achieved. Tears or disruption of the basement membrane can be visualized under the microscope.

### Chemical Decontamination (Step 140)

The amnion is then placed into a sterile Nalgene jar for the next step for additional cleaning. If the chorion is to be recovered and processed further, it too is placed in its own sterile Nalgene jar for additional cleaning. If the chorion is not to be kept or used further, it can be discarded in an appropriate biohazard container. In one aspect, the following procedure can be used to clean the amnion.

Each Nalgene jar is aseptically filled with 18% saline hypertonic solution and sealed (or sealed with a top). The jar is then placed on a rocker platform and agitated for between 30 and 90 minutes, which further cleans the tissue of contaminants. If the rocker platform was not in the critical environment (e.g., the manufacturing hood), the Nalgene jar is returned to the controlled/sterile environment and opened. Using sterile forceps or by aseptically decanting the contents, the tissue is gently removed from the Nalgene jar containing the 18% hypertonic saline solution and placed into an empty Nalgene jar. This empty Nalgene jar with the tissue is then aseptically filled with a premixed antibiotic solution. Preferably, the premixed antibiotic solution is comprised of a cocktail of antibiotics, such as Streptomycin Sulfate and Gentamicin Sulfate. Other antibiotics, such as Polymixin B Sulfate and Bacitracin, or similar antibiotics now available or available in the future, are also suitable. Additionally, it is preferred that the antibiotic solution be at room temperature when added so that it does not change the temperature of or otherwise damage the tissue. This jar or container containing the tissue and antibiotics is then sealed or closed and placed on a rocker platform and agitated for, preferably, between 60 and 90 minutes. Such rocking or agitation of the tissue within the antibiotic solution further cleans the tissue of contaminants and bacteria.

Again, if the rocker platform was not in the critical environment (e.g., the manufacturing hood), the jar or container containing the tissue and antibiotics is then returned to the critical/sterile environment and opened. Using sterile forceps, the tissue is gently removed from the jar or container and placed in a sterile basin containing sterile water or normal saline (0.9% saline solution). The tissue is allowed to soak in place in the sterile water/normal saline solution for at least 10 to 15 minutes. The tissue may be slightly agitated to facilitate removal of the antibiotic solution and any other contaminants from the tissue. After at least 10 to 15 minutes, the tissue is ready to be dehydrated and processed further.

For the chorion layer, the following exemplary procedure can be used. After separation of the chorion from the amnion and removal of clotted blood from the fibrous layer, the chorion is rinsed in 18% saline solution for 30 min. During the first rinse cycle, 18% saline is heated in a sterile container using laboratory heating plate such that the solution temperature is approximately 48° C. The solution is decanted, the chorion tissue is placed into the sterile container, and decanted saline solution is poured into the container. The container is sealed and placed on rocker plate and agitated for 1 hour. After 1 hour agitation bath, remove the tissue and place the tissue into second heated agitation bath for an additional 1 hour rinse cycle. Next, the chorion tissue is placed into 200 ml of 0.5% Triton-X wash solution. The container is sealed and agitated without heat for 2 hours. The tissue is next washed with deionized water (250 ml of DI water×4) with vigorous motion for each rinse. The tissue is removed and placed into a container of 1×PBS w/EDTA solution. The container is sealed and agitated for 1 hour at controlled temperature for 8 hours. The tissue is removed and rinsed using sterile water. A visual inspection was performed to remove any remaining discolored fibrous blood material from the membrane. Membrane should have a cream white visual appearance with no evidence of brownish discoloration.

In one embodiment, the chorion is decellularized. The chorion may be decellularized using any technique, including but not limited to, physical removal using a cell scraper, freezing the membrane, or exposing the membrane to nonionic detergents, anionic detergents, and/or nucleases.

### Preparation of Tissue Graft and Dehydration (Step 150)

After decellularization the amnion membrane is ready to be used to produce the tissue graft. In one aspect, the amnion layer is laid on a suitable drying fixture, where the exposed basement membrane is adjacent to the surface of the drying fixture. In other aspects, the amnion can be placed on the surface of the drying fixture such that the exposed basement membrane is facing up. The drying fixture is preferably sized to be large enough to receive the amnion tissue, fully, in laid out, flat fashion. In one aspect, the drying fixture is made of Teflon or of Delrin, which is the brand name for an acetal resin engineering plastic invented and sold by DuPont and which is also available commercially from Werner Machine, Inc. in Marietta, Ga. Any other suitable material that is heat and cut resistant, capable of being formed into an appropriate shape to receive wet tissue and to hold and maintain textured designs, logos, or text can also be used for the drying fixture. It is desirable that the amnion tissue be placed on the drying fixture so that it completely covers as many “product spaces” (as explained hereinafter) as possible.

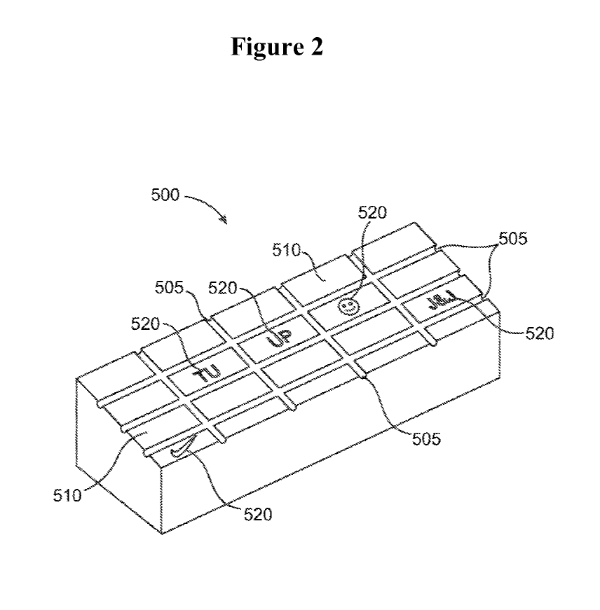

In one aspect, similar to that shown in FIG. 2, the receiving surface of the drying fixture 500 has grooves 505 that define the product spaces 510, which are the desired outer contours of the tissue after it is cut and of a size and shape that is desired for the applicable surgical procedure in which the tissue will be used. For example, the drying fixture can be laid out so that the grooves are in a grid arrangement. The grids on a single drying fixture may be the same uniform size or may include multiple sizes that are designed for different surgical applications. Nevertheless, any size and shape arrangement can be used for the drying fixture, as will be appreciated by those skilled in the art. In another embodiment, instead of having grooves to define the product spaces, the drying fixture has raised ridges or blades.

Within the “empty” space between the grooves or ridges, the drying fixture can include a slightly raised or indented texture in the form of text, logo, name, or similar design 520. This textured text, logo, name, or design can be customized. When dried, the tissue will mold itself around the raised texture or into the indented texture—essentially providing a label within the tissue itself. Preferably, such texture/label can be read or viewed on the tissue in only one orientation so that, after drying and cutting, an end user (typically, a clinician) of the dried tissue will be able to tell the stromal side from the basement side of the dried tissue. The reason this is desired is because, during a surgical procedure, it is desirable to place the allograft in place, with basement side down or adjacent to the native tissue of the patient receiving the allograft. FIG. 2 illustrates a variety of marks, logos, and text 520 that can be included within the empty spaces 510 of the drying fixture 500. Typically, a single drying fixture will include the same design or text within all of the empty spaces; however, FIG. 2 shows, for illustrative purposes, a wide variety of designs that can be included on such drying fixtures to emboss each graft.

After the decellularized amnion layer with substantially no epithelial cells on the surface of the basement membrane has been applied to the drying fixture, a chorion membrane is applied to the base amnion layer to produce the laminated tissue graft.

In one embodiment, micronized placental tissue can be applied between the amnion and chorion layers. Suitable micronized compositions are disclosed in U.S. Patent Publication Nos. 2013-0344162, 2014-0050788-A1, 2014-0052274, which are incorporated herein by reference in their entirety.

Other biodegradable biologically compatible materials can be used to form a multi-layered tissue graft. Examples of such materials include, but are not limited to, amnion and/or chorion that has not been decellularized, micronized amnion and/or chorion, allograft pericardium, allograft acellular dermis, amniotic membrane (i.e., both amnion and chorion), Wharton's jelly ad/or other umbilical cord components, purified xenograft Type-1 collagen, biocellulose polymers or copolymers, biocompatible synthetic polymer or copolymer films, purified small intestinal submucosa, bladder acellular matrix, cadaveric fascia, or any combination thereof.

The actual number of layers will depend upon the surgical need and procedure with which the tissue graft is designed to be used for. For example, in periodontal applications such as root coverage procedures, a single membrane that is between 20-50 μm in thickness to multilayer tissue grafts having a thickness up to 2 mm can be used. In one aspect, the number of membranes laminated to the base amnion tissue with substantially all of the epithelial cells removed can be one, two, five and or ten, with the ultimate number of layers dependent of the type of membrane used, and the expected indication for use.

The multi-layered tissue grafts described herein are thicker and stronger than a single layer of base amnion. The base amnion may have handling limitations. In general, tissue grafts are cut to size to match the morphology of the wound, placed on or within the wound, and if desired, can be held in place with sutures. If left unsupported, the base amnion tissue may fall over itself, and may bunch together. Moreover, the native amnion tissue may be unable to hold sutures. Sutures may tear the tissue during placement, and may be knocked free by the patient. These issues make trimming, placement, and securing a single layer of base amnion tissue less than ideal for the medical professional.

Decellularization of the amnion and/or chorion can be useful in some applications. For example and without being bound by theory, removal of the epithelial layer may allow faster release of bio-active factors that are present in the tissue graft. Decellularization may reduce the immunogenicity of the tissue grafts and/or decrease recovery times. Even though the placenta is normally an immune privileged tissue, lysis of cells present in the tissue grafts may nonetheless result in an inflammatory and/or immune response.

Once the tissue graft is placed on the drying fixture, in one aspect, the drying fixture is placed in a sterile Tyvex (or similar, breathable, heat-resistant, and sealable material) dehydration bag and sealed. Such a breathable dehydration bag prevents the tissue from drying too quickly and prevents cross-contamination, when tissue from multiple donors is dehydrated in the oven at the same time. If multiple drying fixtures are being processed simultaneously, each drying fixture is either placed in its own Tyvex bag or, alternatively, placed into a suitable mounting frame that is designed to hold multiple drying frames thereon and the entire frame is then placed into a larger, single sterile Tyvex dehydration bag and sealed.

The Tyvex dehydration bag containing the one or more drying fixtures is then placed into an oven or incubator (vacuum or non-vacuum) that has been preheated to approximately 35 to 50° C. The Tyvex bag remains in the oven for between 30 and 120 minutes, although approximately 45 minutes at a temperature of approximately 45° C. appears to be ideal to dry the tissue sufficiently but without over-drying the tissue. The specific temperature and time for any specific oven will need to be calibrated and adjusted based on other factors including altitude, size of the oven, accuracy of the oven temperature, material used for the drying fixture, number of drying fixtures being dried simultaneously, whether a single or multiple frames of drying fixtures are dried simultaneously, and the like.

Dehydration can also be performed using a dehydration device as described in U.S. Patent Publication No. 2014-0051059, filed Jan. 17, 2013, which is incorporated herein by reference in its entirety.

In certain aspects the dehydrated tissue graft is not physically altered except for final cutting and packaging (step 160). When completed, the processed tissue graft has a semi-transparent appearance with a whitish coloration. The tissue is pliable to withstand bending and sizing in its dry, non-hydrated state. The tissue grafts described herein can be stored at room temperature for extended periods of time.

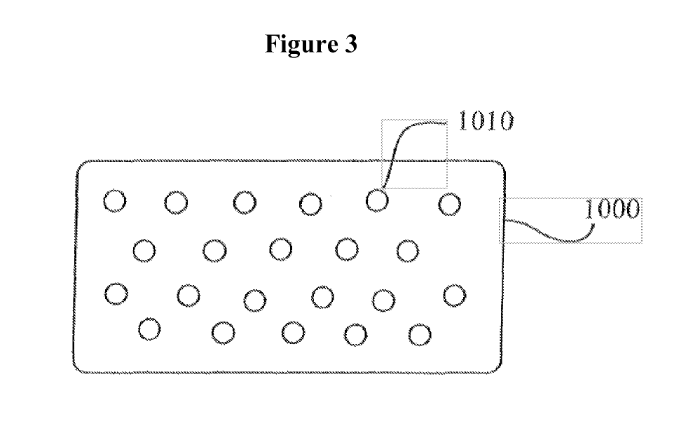

In certain aspects, after the dehydration phase, the tissue graft is perforated using a small gauge needle, or punch to produce a mesh-type configuration, as shown in FIG. 3, where a plurality of holes 1010 are present in graft 1000. The size and spacing of the holes can vary depending upon the application. In one aspect, the holes have a diameter from (0.2 mm-2.0 mm or 0.2 mm to 0.5 mm) and are spaced from 1 mm to 5 mm or 2 mm to 4 mm apart. The holes may facilitate cell migration and provide for the passage of blood and other factors to the opposite side of the graft, which ultimately enhances wound healing. For example, in several perioplastic procedures, the membrane is placed directly onto the exposed root of the tooth. Here, the tooth surface does not supply a source of blood and the holes provide a source of blood and other factors to the side of the membrane that is in direct contact with the tooth surface. In multi-layered products, the holes may allow for a more rapid and even distribution of wound healing agents which may be applied to and/or adsorbed onto the graft prior to implantation to further improve the regenerative potential of the treatment.

### Cutting & Packaging (Step 160)

Once the tissue graft has been adequately dehydrated, the tissue graft is ready to be cut into specific product sizes and appropriately packaged for storage, terminal sterilization, and later surgical use. In one aspect, the Tyvex bag containing the dehydrated tissue is placed back into the sterile/controlled environment. The number of grafts to be produced is estimated based on the size and shape of the tissue on the drying fixture(s). An appropriate number of pouches, one for each tissue graft, is introduced into the sterile/controlled environment. The drying fixture(s) are then removed from the Tyvex bag.

If the drying fixture has grooves, then the following exemplary procedure may be followed for cutting the tissue into product sizes. If the drying fixture is configured in a grid pattern, a #20 or similar straight or rolling blade is used to cut along each groove line in parallel. Then, all lines in the perpendicular direction are cut. Alternatively, if the drying fixture has raised edges or blades, then a sterile roller can be used to roll across the drying fixture. Sufficient pressure must be applied so that the dehydrated tissue is cut along all of the raised blades or edges of the drying fixture.

After cutting, each tissue graft is placed in a respective “inner” pouch. The inner pouch, which preferably has a clear side and an opaque side, should be oriented clear side facing up. The tissue graft is placed in the “inner” pouch so that the texture in the form of text, logo, name, or similar design is facing out through the clear side of the inner pouch and is visible outside of the inner pouch. This process is repeated for each separate graft.

Each tissue graft is then given a final inspection to confirm that there are no tears or holes, that the product size (as cut) is within approximately 1 millimeter (plus or minus) of the specified size for that particular graft, that there are no noticeable blemishes or discoloration of the tissue, and that the textured logo or wording is readable and viewable through the “inner” pouch.

To the extent possible, oxygen is removed from the inner pouch before it is sealed. The inner pouch can be sealed in any suitable manner; however, a heat seal has shown to be effective. In one aspect, after packaging, the product is terminally sterilized by radiation, using gamma or electron beam sterilization with a target dose of 17.5 kGy. Next, each inner pouch is separately packaged in an “outer” pouch for further protection, storage, and shipment.

It should be noted that the above process does not require freezing of the tissue to kill unwanted cells, to decontaminate the tissue graft, or otherwise to preserve the tissue graft. The dehydrated tissue grafts described herein are designed to be stored and shipped at room or ambient temperature without need for refrigeration or freezing.

### Product Release (Step 170)

Before the tissue graft is ready for shipment and release to the end user, all documentation related to manufacture, recovery and donor eligibility are reviewed and deemed acceptable by the quality assurance department and the medical director.

Appropriate labeling and chain of custody is observed throughout all of the above processes, in accordance with accepted industry standards and practice. Appropriate clean room and sterile working conditions are maintained and used, to the extent possible, throughout the above processes.

### Applications of Tissue Grafts

These membranes are composed of at least one layer of amnion tissue where the epithelial layer has been substantially removed in order to expose the basement layer to host cells. By removing the epithelial layer, cells from the host can more readily interact with the cell-adhesion bio-active molecules located onto top and throughout the basement membrane. These grafts could be composed of one or several layers of amnion tissue, combined with chorion and/or other biocompatible membranes to form laminated tissue grafts.

The laminin structure of amnion tissue is nearly identical to that of native human oral mucosa tissue. They both include high level of laminin-5, a cell adhesion factor show to bind gingival epithelia-cells, throughout upper portions of the basement membrane. By creating amnion-chorion or composite grafts where amnion tissue forms the outer most layer of the membrane, the membranes described herein will be highly suited for use in regenerative procedures involving the oral mucosa. Due to the similarity of mucosa tissue found in oral cavity, nose, throat, vagina, and rectum, it should also be highly effective in regenerative procedures in those anatomical regions. The laminated tissue grafts also could be effective in broad range of applications including the treatment of burns, chronic wounds, and as grafts in cardiovascular surgery.

These membranes can be soaked with a bioactive agent such as a solution composed of naturally occurring growth factors sourced from platelet concentrates, either using autologous blood collection and separation products, or platelet concentrates sourced from expired banked blood; bone marrow aspirate; stem cells derived from concentrated human placental cord blood stem cells, concentrated amniotic fluid stem cells or stem cells grown in a bioreactor; or antibiotics. Here, one or more membrane layers of the tissue graft absorb the bioactive agent. Upon application of the wet tissue graft with bioactive agent to the wound, the bioactive agent is delivered to the wound over time.

The tissue grafts are easy to handle and do not readily break. Additionally, the tissue may composed in such a manner as to hold sutures and/or provide specific rates of resorption based on the requirements of the indication in which it is being used.

The tissue grafts described herein have numerous applications and can be used in a variety of procedures. For example, the tissue grafts can be used in a variety of perioplastic procedures, including but not limited to the treatment of recession defects; to increase gingival tissue height and width; to increase the amount of attached gingival tissue at the gingival margin; to increase the zone of attached gingival tissue; elimination of a frenum pull; regeneration of lost patella tissue; repair of the Schneiderian membrane in the sinus cavity; repair of soft tissue around dental implants; vestibuloplasty; and guided tissue regeneration. In oral surgery, the laminated tissue grafts could be used to improve soft tissue outcomes and grow new bone in guided bone regeneration procedures. In applications involving oral mucosa, the tissue grafts can be used for treating mouth sores and oral lesions and/or to replace larger amounts of mucosal tissue lost through disease or traumatic injury. These same membranes could also be used in reconstructive procedures where the tissue is composed of mucosa including in ear, nose, and throat, urogynecology, and surgical procedures involving the rectum and anus.

Throughout this application, various publications are referenced. The disclosures of these publications in their entireties are hereby incorporated by reference into this application in order to more fully describe the compounds, compositions and methods described herein.

Various modifications and variations can be made to the compounds, compositions and methods described herein. Other aspects of the compounds, compositions and methods described herein will be apparent from consideration of the specification and practice of the compounds, compositions and methods disclosed herein. It is intended that the specification and examples be considered as exemplary.

1. A laminated tissue graft comprising:
a first membrane comprising modified decellularized amnion, wherein the modified amnion comprises an exposed basement membrane;
a second membrane comprising cleaned and washed chorion membrane retaining a cellular component layered adjacent to the first membrane, said chorion membrane layered adjacent to said first membrane; and
optionally one or more additional membranes layered over the first or second membrane, wherein the one or more additional membranes is selected from the group consisting of amnion, decellularized amnion, chorion, decellularized chorion, allograft pericardium, allograft acellular dermis, amniotic membrane, Wharton's jelly, and combinations thereof;
wherein the laminated tissue graft is dehydrated.

2. The laminated tissue graft of claim 1, wherein one side of the first membrane is a decellularized fibroblast layer.

3. The laminated tissue graft of claim 1, wherein one side of the first membrane is an exposed compact layer.

4. The laminated tissue graft of claim 1, wherein one side of the first membrane is the other side of the basement membrane, wherein the other side of the basement membrane is exposed.

5. (canceled)

6. The laminated tissue graft of claim 1, further comprising at least one component selected from the group consisting of a naturally occurring growth factor, a platelet concentrate, a bone marrow aspirate, stem cells, or an antibiotic.
